(19) United States
(12) Patent Application Publication
(10) Pub. No.: US 2019/0358344 A1
(43) Pub. Date: Nov. 28, 2019

(54) MINERAL COATED MICROPARTICLES FOR SUSTAINED DELIVERY OF STEROIDS

(71) Applicant: Dianomi Therapeutics, Inc., Parker, CO (US)

(72) Inventors: Anna Clements, Madison, WI (US); William L. Murphy, Waunakee, WI (US); Barry Kurokawa, Parker, CO (US)

(21) Appl. No.: 16/419,545

(22) Filed: May 22, 2019

Related U.S. Application Data
(60) Provisional application No. 62/675,612, filed on May 23, 2018.

(51) Int. Cl. A61K 47/69; A61K 9/00; A61K 9/50

(52) U.S. Cl. CPC A61K 47/6927; A61K 9/5073; A61K 9/0019; A61K 47/6923

(57) ABSTRACT

Disclosed are formulations for providing a steroid. Formulations include a mineral coated microparticles wherein a steroid is adsorbed to the mineral coating or incorporated within the mineral coating. Also disclosed are methods for sustained delivery of a steroid and methods for treating inflammation or pain using a formulation for providing sustained delivery of a steroid.

### BACKGROUND

Synthetic steroids are a class of pharmaceutical drugs that are used to treat a variety of medical conditions. Often, treatment involves systemic administration of steroids through oral, inhaled, intravenous, or subcutaneous routes. Corticosteroids, in particular, are used is in the practice of pain management because of their anti-inflammatory properties. Topical formulations are also used to treat local inflammation. However, steroids can have many undesired effects including, but not limited to, hypertension, hypokalemia, hypernatremia, metabolic alkalosis, immunosuppression, secondary infection, and permanent eye damage. There is a need for a technology that can deliver the benefits of steroid therapeutics without the undesirable side effects.

### BRIEF DESCRIPTION

The present disclosure is directed to formulations for providing sustained delivery of steroids. Formulations include a mineral coating on a substrate material wherein a steroid is adsorbed to the mineral coating. Also disclosed are methods for sustained delivery of a steroid and methods for treating inflammatory diseases or pain using a formulation for providing sustained delivery of a steroid.

Controlled delivery of steroids can improve their clinical benefit and decrease their off-target or detrimental side effects. Many steroids have short plasma half-lives (on the order of hours), so frequent repeat administration is required to maintain therapeutic levels. The sustained delivery technology for therapeutic molecules described herein, including steroids, maintain a therapeutic concentrations of the molecule, either systemically or locally, by releasing the molecule over a prolonged period of time. The sustained delivery technology releases the molecule upon dissolution or degradation.

Delivery approaches for steroids disclosed herein include localized delivery and systemic delivery approaches. For systemic delivery approaches, the steroid is administered with the intent of elevating the serum concentration of the steroid. Systemic delivery of steroids can be achieved by providing a depot which releases steroid which then enters circulation. In localized delivery, the steroid is active at the site of interest and does not impact regions outside of the site of interest.

Previous strategies to prolong the benefit of therapeutic molecules through sustained or localized delivery often resulted in reduced activity and/or required higher doses. Further, the loading capacity of materials used to provide sustained delivery can limit the achievable dose of steroid when administered to a patient. Accordingly, there exists a need for alternative delivery systems that can provide sustained delivery of active steroids at relevant doses.

In one aspect, the present disclosure is directed to a formulation for providing a steroid. The formulation includes a mineral coated microparticle comprising a microparticle core; and mineral coating on the microparticle core; and a steroid. Mineral coatings have the ability to steroids through electrostatic interactions between the charged and polar groups of the steroid and the charged ions in the mineral structure. Alternatively, steroid molecules can become “trapped” in the porous mineral coating structure. Steroids are then released upon coating dissolution. Incorporation of different ions within the mineral coating can impact mineral coating nanostructure and dissolution rate. Mineral coatings can, therefore, be tailored to best suit the delivery of particular steroid at a specific release rate. Further, the nanostructure of the coating can be tailored to preserve specific steroid activity by stabilizing the 3-dimensional structure until release.

The mineral coating is formed on the microparticle core to form a mineral coated microparticle. In one embodiment, the mineral coating is formed by precipitation of the coating onto the core. The core material can be selected from particles made from many different materials including ceramics, metals, minerals, oxides, polymers, or composites. The core may also be a nucleation site of critical size for coating precipitation. Mineral precipitates on the core from an ionic solution to form a mineral coating. In one embodiment, multiple layers of mineral coating are formed on the core. In one embodiment, the layers are composed of the same mineral composition, while in other embodiments the layers are composed of different mineral compositions. In one embodiment, the mineral coating precipitated on the core material is a calcium phosphate mineral. In one embodiment, the mineral coating is nanostructured.

The formulation also includes a steroid. In one embodiment, a steroid is adsorbed to the mineral coating on the mineral coated microparticle. In another embodiment, the steroid is incorporated throughout the mineral coating on the mineral coated microparticle. In another embodiment, there are multiple layers of coating which can each incorporate steroid or other compounds.

In one embodiment, the steroid is released from the mineral coating upon coating dissolution. The dissolution kinetics can therefore be changed to alter the release kinetics of the steroid. In one embodiment, the mineral coating is a calcium phosphate mineral, the dissolution kinetics of which can be altered by incorporating substitution ions within the mineral. In one embodiment, the substitution ions are incorporated during coating formation. In some embodiments, the substitution ions make the mineral coating more soluble while in other embodiments the substitution ions make the mineral coating less soluble.

In one embodiment, the formulation also includes a carrier for the mineral coated microparticles. The carrier can be, but is not limited to, a solution, a gel, a liquid, a solid, or a gas. In one embodiment, the carrier contains an active agent. The active agent can be, but is not limited to, a protein, a small molecule, a steroid, an anti-inflammatory agent, or a drug. In another embodiment, a second active agent is also incorporated in or adsorbed to the mineral coating.

In one aspect, the present disclosure is directed to a method for immediate and sustained delivery of a steroid. The method includes providing a formulation to an individual in need thereof, the formulation including a mineral coated microparticle and a steroid. In one embodiment, the steroid is adsorbed to the mineral coating. In another embodiment, the steroid is incorporated throughout the mineral coating. In one embodiment, the formulation includes a carrier for the mineral coated microparticle. In one embodiment, the carrier can contain an active agent to improve delivery of the steroid. The active agent can be selected from, but is not limited to, a protein, a small molecule, or a drug. In another embodiment, the carrier can contain a steroid for immediate activity. In another embodiment, the carrier may contain multiple active agents.

In one embodiment, the method for immediate and sustained delivery of a steroid involves administration of a formulation comprising a mineral coated microparticle and a steroid. The formulation can be administered by, but is not limited to, subcutaneous injection, local injection, inhalation, oral administration, or topical application. In one embodiment, the formulation is locally injected into a tissue for local delivery of steroid. In another embodiment, the formulation is injected subcutaneously or into a tissue for systemic administration of steroid. In one embodiment, the formulation acts as a delivery depot to increase the systemic concentration of steroid. In one embodiment, the formulation is injected intra-articularly in a joint to deliver steroids into the synovial fluid. In another embodiment, the formulation is injected in the synovial membrane to deliver the steroid to the synovium.

In one aspect, the present disclosure is directed to a method for treating inflammation in a subject in need thereof. The method includes administering a formulation to the subject, wherein the formulation comprises a mineral coated microparticle and a steroid. Particularly useful steroids for treating inflammation are corticosteroids. In one embodiment, the formulation is administered locally at the site of local inflammation. In another embodiment, the formulation is administered for systemic delivery of steroid to control local or systemic inflammation. In one embodiment, the formulation acts as a delivery depot to increase the systemic concentration of steroid. In one embodiment, the inflammation is associated with osteoarthritis. In one embodiment, the formulation is administered locally to a joint to treat inflammation associated with osteoarthritis. In another embodiment, the formulation is used to treat inflammation caused by acute injury.

In one aspect, the present disclosure is directed to a method for treating pain in a subject in need thereof. The method includes administering a formulation to the subject, wherein the formulation comprises a mineral coated microparticle and a steroid. Particularly useful steroids for treating pain are corticosteroids. In one embodiment, the formulation is administered locally at the site of pain. In another embodiment, the formulation is administered for systemic delivery of steroid to control pain. In one embodiment, the formulation acts as a delivery depot to increase the systemic concentration of steroid. In one embodiment, the pain is caused by osteoarthritis. In one embodiment, the formulation is administered to a joint to treat pain caused by osteoarthritis

### BRIEF DESCRIPTION OF THE DRAWINGS

FIG. 1A-C are schematics representing different approaches for mineral coated microparticle fabrication and representative cross sections of the microparticle at each step. FIG. 1 A depicts the fabrication of mineral coated microparticles in which steroid is adsorbed to the mineral coating after coating formation. FIG. 1B depicts the fabrication of mineral coated microparticles which incorporate steroid during coating formation and incorporate steroid throughout the coating. FIG. 1C depicts the fabrication of layered mineral coated microparticles and loading of steroid on each layer of mineral coating.

### DETAILED DESCRIPTION

The nano-structured calcium phosphate mineral coatings disclosed herein provide a platform for sustained delivery of steroids. The mineral coated microparticles offer an injectable and localized delivery system that can lower the dose and off-target side-effects when compared to bolus injection of steroids, particularly with steroids having short half-lives or having reduced activity when modified such as by encapsulation and/or making fusion products. The formulations and methods disclosed herein advantageously allow for both immediate effect of the steroid that is delivered in unbound form, as well as sustained effect of the steroid by adsorbing the steroid to mineral coated microparticles that provide sustained delivery of the steroid as the mineral coating degrades and releases the steroid. Other active agents can be incorporated into the mineral coated microparticles or within a carrier solution to improve the delivery of the steroid.

Mineral coated microparticles offer a delivery system that can sustainably release steroids while maintaining their activity. Further, these microparticles remain localized when injected in vivo and offer a localized delivery system for steroids which can allow for lower therapeutic dosages when compared to systemic subcutaneous or intravenous delivery. Localized delivery of steroids can also limit their off-target effects. Further, release of steroid from mineral coated microparticles can be easily tailored by altering the coating composition. In addition, mineral coated microparticles have a high binding capacity for steroids which allows them to sustainably deliver a suitable dose of steroid with little delivery system material. This may widen the applicability of sustained delivery systems for steroids.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements

The present disclosure is directed to formulations for providing a steroid. In some embodiments, formulations include mineral coated microparticles wherein a steroid is adsorbed to the mineral coating. In some embodiments, formulations include mineral coated microparticles wherein a steroid is incorporated throughout the mineral coating. In some embodiments, the mineral coating has multiple layers. In some embodiments, steroid is adsorbed on multiple layers of the mineral coating. In some embodiments, formulations include a carrier and mineral coated microparticles wherein a steroid is adsorbed to the mineral coating. In some embodiments, the carrier contains an active agent. In some embodiments, the active agent is a steroid. In some embodiments, the active agent is an anti-inflammatory. In some embodiments, formulations include a carrier including an active agent and mineral coated microparticles wherein an active agent is incorporated within the mineral coating and wherein an active agent is adsorbed to the mineral coating. Active agents included in the carrier provide a rapid effect following administration whereas active agent adsorbed to the mineral coating and/or incorporated within the mineral coating provides a sustained delivery as the mineral coating degrades. Also disclosed are methods for sustained delivery of a steroid and methods for treating inflammation or pain using a formulation for providing sustained delivery of a steroid.

In one aspect, the present disclosure is directed to a formulation for providing a steroid. Particularly useful steroids include sex hormones, corticosteroids, glucocorticoids, mineralocorticoids, and anabolic steroids. Corticosteroids are particularly useful for treating inflammatory conditions and pain. Particularly useful corticosteroids include Class B corticosteroids, including Triamcinolone acetonide and pharmaceutically acceptable salts thereof. Class A (including prednisolone and pharmaceutical equivalents), Class C (including betamethasone and pharmaceutical equivalents), or Class D (fluticasone or fluticasone propionate and pharmaceutical equivalents) corticosteroids are also particularly useful.

In one embodiment, the formulation includes a mineral coated microparticle, wherein the mineral coated microparticle comprises a core; a mineral coating on the core; and a steroid. In one embodiment, the core is a nucleation site for coating precipitation. In one embodiment, the steroid is adsorbed to the mineral coating. In one embodiment only the steroid is incorporated throughout the mineral coating. In one embodiment, there are layers of mineral coating on the core. In one embodiment, the steroid is adsorbed to multiple layers of mineral coating on the core. In one embodiment, multiple, different active agents are adsorbed to the mineral coating along with a steroid. In one embodiment, multiple steroids are adsorbed to the mineral coating. The steroids can be different types of steroids. In one embodiment, other anti-inflammatory agents are adsorbed to the mineral coating along with one or multiple steroids.

In one embodiment, the formulation includes a mineral coated microparticle, wherein the mineral coated microparticle comprises a core, a first layer of mineral coating on the core, an active agent adsorbed onto the first layer of mineral coating, a second layer of mineral coating and a second active agent adsorbed to the second layer of mineral coating. In one embodiment, the active agent adsorbed onto the first layer of mineral coating is the same as the active agent adsorbed on the second layer of mineral coating. In another embodiment, the active agent adsorbed onto the first layer of mineral coating is different than the active agent adsorbed on the second layer of mineral coating. In one embodiment, more than one active agent is adsorbed on each layer of mineral coating (e.g., 2, 3, 4, 5, 6, 7, 8, 9, 10, or more active agents). In one embodiment, at least one of the active agents adsorbed on any of the layers of mineral coating is a steroid. In one embodiment, all of the active agents adsorbed on the layers of mineral coating are steroids.

In one embodiment, the formulation includes a mineral coated microparticle, wherein the mineral coated microparticle comprises a core, a plurality of layers of mineral coating, and active agents. At least one of the active agents is a steroid. The layers of mineral coating can be the same coating formulations as described herein. The layers of mineral coating can also be different coating formulations as described herein. The active agents can be adsorbed onto the layers of mineral coating after each layer of mineral coating is prepared as described herein. The active agents can be incorporated within the layers of mineral coating during mineral formation as described herein. The active agents can be the same active agent as described herein. The active agents can be different active agents as described herein. At least one active agent is a steroid.

The term “formulation”, as used herein, generically indicates the beneficial agents and mineral coated microparticles are formulated, mixed, added, dissolved, suspended, solubilized, formulated into a solution, carried and/or the like in or by the fluid, gas, or solid in a physical-chemical form acceptable for patient administration.

In one embodiment, the formulation includes a carrier, wherein the carrier is for a mineral coated microparticle, wherein the mineral coated microparticle comprises a core; a mineral coating on the core; and a steroid adsorbed to the mineral coating. In one embodiment, another active agent is adsorbed to the mineral coating along with the steroid. In one embodiment, the carrier is a liquid. In one embodiment, the carrier is a solution or a liquid. In another embodiment, the carrier is a gel. In yet another embodiment the carrier is a gas. In yet another embodiment the carrier is a solid. In one embodiment, the carrier contains an active agent. In one embodiment, the active agent is a steroid. In one embodiment, the active agent in the carrier contains the same steroid adsorbed on or incorporated within the mineral coating. In one embodiment, the active agent in the carrier is a different steroid than the steroid adsorbed on or incorporated within the mineral coating. In one embodiment, the carrier contains more than one active agent. In one embodiment, the carrier contains multiple steroids. In another embodiment, the carrier contains a steroid and one or more active agents that are not steroids. In one embodiment, the carrier contains an anti-inflammatory.

In one embodiment, the at least one of the active agents adsorbed to the mineral coating is the same as the active agent in the carrier. In another embodiment, the active agents adsorbed to the mineral coating are all different from the active agent in the carrier. In another aspect, at least two different active agents are adsorbed to the mineral coating. Contemplated embodiments further include 2, 3, 4, 5 or more different active agents adsorbed to the mineral coating. In one embodiment, the active agent incorporated within the mineral coating is the same as the active agent in the carrier. In another embodiment, the active agent incorporated within the mineral coating is different from the active agent in the carrier. In another aspect, at least two different active agents are incorporated within the mineral coating. Contemplated embodiments further include 2, 3, 4, 5 or more different active agents incorporated within the mineral coating. At least one of the active agents is a steroid. In another aspect, an active agent can be incorporated within the mineral coating in combination with an active agent adsorbed to the mineral coating. Formulations include 2, 3, 4, 5 or more different active agents in the carrier solution.

Particularly suitable active agents can be anti-inflammatory molecules. Anti-inflammatory molecules may be proteins, small molecules, hormones, cytokines, antibodies, non-steroidal anti-inflammatories, or steroids.

When formulated in one formulation the unbound active agent contained in the carrier and the active agent adsorbed to the mineral coated microparticle have profiles of action that are identical or substantially identical to the profiles of action when the unbound active agent and the active agent adsorbed to the mineral coated microparticle are administered in separate formulations. Thus, the unbound active agent functions as a bolus administration with rapid or immediate profile of action whereas the bound active agent (adsorbed to the mineral coated microparticle) functions as a sustained release profile of action.

As used herein, an “effective amount”, a “therapeutically effective amount”, a “prophylactically effective amount” and a “diagnostically effective amount” is the amount of the unbound active agent and the active agent adsorbed to the mineral coated microparticle needed to elicit the desired biological response following administration.

Suitable liquid carriers include water, saline, isotonic saline, phosphate buffered saline, Ringer's lactate, and the like. Suitable gel carriers include collagen, hydrogels, polymer gels, polyethylene glycol, and the like.

Formulations can also include other components such as surfactants, preservatives, and excipients. Surfactants can reduce or prevent surface-induced aggregation of the active agent and the mineral coated microparticles. Various conventional surfactants can be employed, such as polyoxyethylene fatty acid esters and alcohols, and polyoxyethylene sorbitol fatty acid esters. Amounts will generally range from about 0.001 and about 4% by weight of the formulation. Pharmaceutically acceptable preservatives include, for example, phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) and mixtures thereof. The preservative can be present in concentrations ranging from about 0.1 mg/ml to about 20 mg/ml, including from about 0.1 mg/ml to about 10 mg/ml. A preservative can be used in pharmaceutical compositions such as, but not limited to those described in “Remington: The Science and Practice of Pharmacy, 19th edition, 1995”. Formulations can include suitable buffers such as sodium acetate, glycylglycine, HEPES (4-(2-hydroxyethyl)-1-piperazineethanesulfonic acid) and sodium phosphate. Excipients include components for tonicity adjustment, antioxidants, and stabilizers as commonly used in the preparation of pharmaceutical formulations. Other inactive ingredients include, for example, L-histidine, L-histidine monohydrochloride monohydrate, sorbitol, polysorbate 80, sodium citrate, sodium chloride, and EDTA disodium.

Any suitable material can be used as the core upon which the mineral coating is formed. Particularly suitable core materials are those materials known to be non-toxic to humans and animals. Particularly suitable core materials also include those materials known to degrade and/or dissolve in humans and animals. Suitable core materials include β-tricalcium phosphate (β-TCP), hydroxyapatite (HA), poly lactic co-glycolic acid (PLGA), and combinations thereof β-tricalcium phosphate cores are particularly suitable as the β-tricalcium phosphate degrades rapidly after mineral coating dissolution. Both β-tricalcium phosphate and hydroxyapatite are also particularly suitable cores because they dissolve into calcium and phosphate ions which are easily metabolized by the body. In other embodiments, the core material can be dissolved following mineral coating formation. In other embodiments, the core material is non-degradable.

The mineral coating includes calcium, phosphate, carbonate, and combinations thereof. To prepare a mineral coated microparticle a core material is incubated in a modified simulated body fluid. Simulated body fluid contains the same ion constituents at the same concentrations as human blood plasma. Modified simulated body fluid contains similar, but altered ion constituents as human blood plasma. In some embodiments, the modified simulated body fluid contains twice the concentration of calcium and phosphate as human blood plasma along with the other ionic components of human blood plasma at physiological concentrations. The modified simulated body fluid includes calcium and phosphate, which form the mineral coating on the surface of the core, which results in the mineral coated microparticle. Because the modified simulated body fluid contains a supersaturation of calcium and phosphate, a mineral coating precipitates from solution onto the core material to form the mineral coating. Different mineral coating morphologies can be achieved by varying the amounts and ratios of calcium, phosphate, and carbonate in the modified simulated body solution during coating precipitation. Other ions, or dopants, can also be added to the modified simulated body fluid during coating formation to change the coating composition and/or morphology. Different mineral coating morphologies include, for example, plate-like structure, spherulite-like structure. High carbonate concentration results in a mineral coating having a plate-like structure. Low carbonate concentration results in a mineral coating having a spherulite-like structure. The mineral coating morphology also affects adsorption of the active agent. The mineral coating morphology also affects the preservation of activity of the active agent release from the mineral coating.

Suitable core materials on which the mineral coating is formed include polymers, ceramics, metals, glass and combinations thereof in the form of particles. Suitable particles can be, for example, agarose beads, latex beads, magnetic beads, polymer beads, ceramic beads, metal beads (including magnetic metal beads), glass beads and combinations thereof. The microparticle includes ceramics (e.g., hydroxyapatite, beta-tricalcium phosphate (beta-TCP, β-TCP), magnetite, neodymium), plastics (e.g., polystyrene, poly-caprolactone), hydrogels (e.g., polyethylene glycol; poly(lactic-co-glycolic acid), and the like, and combinations thereof. Particularly suitable core materials are those that dissolve in vivo such as, for example, beta-tricalcium phosphate (beta-TCP, β-TCP).

Suitable microparticle sizes can range from about 1 μM to about 100 μM in diameter. Microparticle diameter can be measured by, for example, measurements taken from microscopic images (including light and electron microscopic images), filtration through a size-selection substrate, and the like.

The modified simulated body fluid (mSBF) for use in the methods of the present disclosure typically includes from about 5 mM to about 12.5 mM calcium ions, including from about 7 mM to about 10 mM calcium ions, and including about 8.75 mM calcium ions; from about 2 mM to about 12.5 mM phosphate ions, including from about 2.5 mM to about 7 mM phosphate ions, and including from about 3.5 mM to about 5 mM phosphate ions; and from about 4 mM to about 100 mM carbonate ions.

In some embodiments, the mSBF can further include about 145 mM sodium ions, from about 6 mM to about 9 mM potassium ions, about 1.5 mM magnesium ions, from about 150 mM to about 175 mM chloride ions, about 4 mM HCO₃⁻, and about 0.5 mM SO₄²⁻ ions.

The pH of the mSBF can typically range from about 4 to about 7.5, including from about 5.3 to about 6.8, including from about 5.7 to about 6.2, and including from about 5.8 to about 6.1.

Suitable mSBF can include, for example: about 145 mM sodium ions, about 6 mM to about 9 mM potassium ions, about 5 mM to about 12.5 mM calcium ions, about 1.5 mM magnesium ions, about 150 mM to about 175 mM chloride ions, about 4.2 mM HCO₃⁻, about 2 mM to about 5 mM HPO₄²⁻ ions, and about 0.5 mM SO₄²⁻ ions. The pH of the simulated body fluid may be from about 5.3 to about 7.5, including from about 6 to about 6.8.

In one embodiment, the mSBF may include, for example: about 145 mM sodium ions, about 6 mM to about 17 mM potassium ions, about 5 mM to about 12.5 mM calcium ions, about 1.5 mM magnesium ions, about 150 mM to about 175 mM chloride ions, about 4.2 mM to about 100 mM HCO₃⁻, about 2 mM to about 12.5 mM phosphate ions, and about 0.5 mM SO₄²⁻ ions. The pH of the simulated body fluid may be from about 5.3 to about 7.5, including from about 5.3 to about 6.8.

In another embodiment, the mSBF includes: about 145 mM sodium ions, about 6 mM to about 9 mM potassium ions, from about 5 mM to about 12.5 mM calcium ions, about 1.5 mM magnesium ions, about 60 mM to about 175 mM chloride ions, about 4.2 mM to about 100 mM HCO₃⁻, about 2 mM to about 5 phosphate ions, about 0.5 mM SO₄²⁻ ions, and a pH of from about 5.8 to about 6.8, including from about 6.2 to about 6.8.

In yet another embodiment, the mSBF includes: about 145 mM sodium ions, about 9 mM potassium ions, about 12.5 mM calcium ions, about 1.5 mM magnesium ions, about 172 mM chloride ions, about 4.2 mM HCO₃⁻, about 5 mM to about 12.5 mM phosphate ions, about 0.5 mM SO₄²⁻ ions, from about 4 mM to about 100 mM CO₃²⁻, and a pH of from about 5.3 to about 6.0.

In embodiments that include a layered mineral coating, a core is incubated in a formulation of modified simulated body fluid. The layer of mineral coating forms on the core during the incubation period of minutes to days. After the initial layer of mineral coating is formed on the core, the mineral coated microparticle can be removed from the modified simulated body fluid and washed. To form a plurality of layers of mineral coating a mineral coated microparticle is incubated in a second, third, fourth, etc. modified simulated body fluid until the desired number of layers of mineral coating is achieved. During each incubation period a new layer of mineral coating forms on the previous layer. These steps are repeated until the desired number of layers of mineral coating is achieved.

During mineral formation, active agents can be included in the modified simulated body fluid to incorporate active agents within the layer of mineral coating during mineral formation. The active agent can be a steroid or can be a different active agent. Following formation of each layer of mineral, the mineral coated microparticle can then incubated in a carrier comprising at least one active agent to adsorb the agent to the layer of mineral coating. After incorporating an active agent within a layer of mineral coating and/or adsorbing an active agent to a layer of mineral coating, another layer of mineral coating can be formed by incubating the microparticle in another formulation of modified simulated body fluid. If desired, layers of mineral coating can incorporate an active agent in the mineral, layers can have an active agent adsorbed to the layer of mineral, the layer of mineral coating can be formed without incorporating an active agent or adsorbing an active agent, and combinations thereof. Mineral coated microparticles having different layers of mineral coating can be prepared by forming a layer of mineral using one formulation of modified simulated body fluid, then incubating the mineral coated microparticle in a different formulation of modified simulated body fluid. Thus, mineral coated microparticles can be prepared to have a plurality of layers of mineral coating wherein each layer is different. Embodiments are also contemplated that include two or more layers of mineral coating that are the same combined with one or more layers of mineral coating that are the different. One of the active agents is a steroid.

Tailoring the composition of the mineral coating in the different layers advantageously allows for tailored release kinetics of the active agent or active agents from each layer of the mineral coating.

In embodiments where incorporation of one or more active agents within the mineral coating is desired, the active agent is included in the mSBF. As mineral formation occurs, active agents become incorporated with the mineral coating.

In other embodiments, magnetic material can be incorporated into mineral coatings. For example, superparamagnetic iron oxide linked to bovine serum albumin can be incorporated into mineral coatings. Linked proteins (e.g., bovine serum albumin) can adsorb onto the mineral coating to incorporate the magnetic material with the mineral coating.

In some embodiments, the mineral coating further includes a dopant. Suitable dopants include halogen ions, for example, fluoride ions, chloride ions, bromide ions, and iodide ions. The dopant(s) can be added with the other components of the mSBF prior to incubating the substrate in the mSBF to form the mineral coating. The dopant ions can alter the dissolution kinetics of the mineral and can thus alter the release kinetics of steroid or other active agent from the mineral coating.

In some embodiments, the halogen ions include fluoride ions. Suitable fluoride ions can be provided by fluoride ion-containing agents such as water soluble fluoride salts, including, for example, alkali and ammonium fluoride salts. Incorporation of fluoride alters the stability of the mineral coating.

The fluoride ion-containing agent is generally included in the mSBF to provide an amount of up to 100 mM fluoride ions, including from about 0.001 mM to 100 mM, including about 0.01 mM to about 50 mM, including from about 0.1 mM to about 15 mM, and including about 1 mM fluoride ions.

It has been found that the inclusion of one or more dopants in the mSBF results in the formation of a halogen-doped mineral coating that can have significantly different morphologies and/or dissolution and release kinetics. The different morphology may be beneficial for preserving the activity of the active agent release from the mineral coating. The control of mineral coating dissolution can be beneficial when tailoring the coating to have desired release kinetics for the active agent to enhance efficacy.

In yet more embodiments, magnetic materials, including magnetite, magnetite-doped plastics, and neodymium, are used for the microparticle core material. Including magnetic materials results in the formation of MCM for which location and/or movement/positioning of the MCM by application of a magnetic force is enabled. The alternate use of magnetic microparticle core materials allows for spatial control of where the active agent and/or the steroid is delivered.

The mineral coatings may be formed by incubating the substrate with the mSBF at a temperature of about 37° C. for a period of time ranging from about 3 days to about 10 days.

After completing the mineral coating preparation, the mineral coatings can be analyzed to determine the morphology and composition of the mineral coatings. The composition of the mineral coatings can be analyzed by energy dispersive X-ray spectroscopy, Fourier transform infrared spectrometry, X-ray diffractometry, and combinations thereof. Suitable X-ray diffractometry peaks can be, for example, at 26° and 31°, which correspond to the (0 0 2) plane, the (2 1 1) plane, the (1 1 2) plane, and the (2 0 2) plane for the hydroxyapatite mineral phase. Particularly suitable X-ray diffractometry peaks can be, for example, at 26° and 31°, which correspond to the (0 0 2) plane, the (1 1 2) plane, and the (3 0 0) plane for carbonate-substituted hydroxyapatite. Other suitable X-ray diffractometry peaks can be, for example, at 16°, 24°, and 33°, which correspond to the octacalcium phosphate mineral phase. Suitable spectra obtained by Fourier transform infrared spectrometry analysis can be, for example, a peak at 450-600 cm⁻¹, which corresponds to O—P—O bending, and a peak at 900-1200 cm⁻¹, which corresponds to asymmetric P—O stretch of the PO₄³⁻ group of hydroxyapatite. Particularly suitable spectra peaks obtained by Fourier transform infrared spectrometry analysis can be, for example, peaks at 876 cm⁻¹, 1427 cm⁻¹, and 1483 cm⁻¹, which correspond to the carbonate (CO₃²⁻) group. The peak for HPO₄²⁻ can be influenced by adjusting the calcium and phosphate ion concentrations of the mSBF used to prepare the mineral coating. For example, the HPO₄²⁻ peak can be increased by increasing the calcium and phosphate concentrations of the mSBF. Alternatively, the HPO₄²⁻ peak can be decreased by decreasing the calcium and phosphate concentrations of the mSBF. Another suitable peak obtained by Fourier transform infrared spectrometry analysis can be, for example, a peak obtained for the octacalcium phosphate mineral phase at 1075 cm⁻¹, which can be influenced by adjusting the calcium and phosphate ion concentrations in the simulated body fluid used to prepare the mineral coating. For example, the 1075 cm⁻¹ peak can be made more distinct by increasing the calcium and phosphate ion concentrations in the simulated body fluid used to prepare the mineral coating. Alternatively, the 1075 cm⁻¹ peak can be made less distinct by decreasing the calcium and phosphate ion concentrations in the simulated body fluid used to prepare the mineral coating.

Energy dispersive X-ray spectroscopy analysis can also be used to determine the calcium/phosphate ratio of the mineral coating. For example, the calcium/phosphate ratio can be increased by decreasing the calcium and phosphate ion concentrations in the mSBF. Alternatively, the calcium/phosphate ratio may be decreased by increasing the calcium and phosphate ion concentrations in the mSBF. Analysis of the mineral coatings by energy dispersive X-ray spectroscopy allows for determining the level of carbonate (CO₃²⁻) substitution for PO₄³⁻ and incorporation of HPO₄²⁻ into the mineral coatings. Typically, the mSBF includes calcium and phosphate ions in a ratio ranging from about 10:1 to about 0.2:1, including from about 2.5:1 to about 1:1.

Further, the morphology of the mineral coatings can be analyzed by scanning electron microscopy, for example. Scanning electron microscopy can be used to visualize the morphology of the resulting mineral coatings. The morphology of the resulting mineral coatings can be, for example, a spherulitic microstructure, plate-like microstructure, and/or a net-like microstructure. Suitable average diameters of the spherulites of a spherulitic microstructure can range, for example, from about 2 μm to about 42 μm. Particularly suitable average diameters of the spherulites of a spherulitic microstructure can range, for example, from about 2 μm to about 4 In another embodiment, particularly suitable average diameters of the spherulites of a spherulitic microstructure can range, for example, from about 2.5 μm to about 4.5 In another embodiment, particularly suitable average diameters of the spherulites of a spherulitic microstructure can range, for example, from about 16 μm to about 42 μm.

Mineral coated microparticles can be stored for later use, washed and stored for later use, washed and immediately used for the adsorption step, or immediately used for the adsorption step without washing. Storage of mineral coated microparticles can include lyophilization.

To adsorb the steroid to the mineral coated microparticle, the mineral coated microparticles are contacted with a solution containing the steroid. This contact can form a steroid loaded mineral coated microparticle. Other active agent(s) can also be adsorbed to the mineral coating along with the steroid by including them in the solution with the steroid. Alternatively, the microparticles can be contacted with a second solution containing other active agent(s) after loading with the steroid. Addition of other active agents can make the delivery of steroid more efficient or effective. In some embodiments, only a steroid is incorporated, adsorbed, or loaded onto or into the mineral coating. As used herein, “active agent” refers to biologically active molecule. As used herein, “steroid loaded mineral coated microparticle” refers to a mineral coated microparticle which has steroid adsorbed to the mineral coating and/or has steroid incorporated throughout the coating. The steroid and/or other active agent(s) can be contacted with the mineral coated microparticle using any suitable method. For example, a solution of the steroid and/or other active agent(s) can be pipetted, poured, or sprayed onto the mineral coated microparticle. Alternatively, the mineral coated microparticle can be dipped in a solution including steroid and/or other active agent(s) along with the steroid. Alternatively, the mineral coated microparticle can be bathed or incubated in a solution containing steroid and/or other active agent(s). The steroid, and/or other active agent(s) adsorb to the mineral coating by an electrostatic interaction between the steroid or active agent and the mineral coating of the mineral coated microparticle. Suitable active agents include biological molecules. Particularly suitable active agents include proteins, small molecules, hormones, steroids, steroids, NSAIDs, cytokines, therapeutic proteins, antibodies, receptor antagonists, or the like. Adsorption of the steroid, or other active agents along with the steroid, to the mineral coated microparticles can be tailored by changing the mineral constituents (e.g., high carbonate and low carbonate microspheres), by changing the amount of mineral coated microparticles incubated with the steroid, or other active agents along, by changing the concentration of steroid, or other active agents in the incubation solution, and combinations thereof.

The steroid and/or other active agent(s) adsorbed to the mineral coating of the mineral coated microparticle are released as the mineral coating degrades. Mineral degradation can be controlled such that the mineral coating can degrade rapidly or slowly. Mineral coating dissolution rates can be controlled by altering the mineral coating composition. For example, mineral coatings that possess higher carbonate substitution degrade more rapidly. Mineral coatings that possess lower carbonate substitution degrade more slowly. Incorporation of dopants, such as fluoride ions, may also alter dissolution kinetics. Alterations in mineral coating composition can be achieved by altering ion concentrations in the modified simulated body fluid during coating formation. Modified simulated body fluid with higher concentrations of carbonate, 100 mM carbonate for example, results in coatings which degrade more rapidly than coatings formed in modified simulated body fluid with physiological carbonate concentrations (4.2 mM carbonate).

To incorporate the steroid and/or other active agent(s) within the mineral coated microparticle, steroids and/or active agent(s) are included in the modified simulated body fluid during the mineral coating process. Particularly suitable active agents include proteins, small molecules, hormones, steroids, steroids, NSAIDs, cytokines, therapeutic proteins, antibodies, receptor antagonists, or the like.

To adsorb steroid, and/or other active agent(s) along with the steroid on different layers of the mineral coated microparticle, mineral coated microparticles are incubated in a solution containing the steroid and/or active agent(s) after the formation of each layer. Some layers may have no active agent and/or no steroid adsorbed onto the surface. Particularly suitable active agents include proteins, small molecules, hormones, steroids, steroids, NSAIDs, cytokines, therapeutic proteins, antibodies, receptor antagonists, or the like.

The steroid can be any type of steroid including sex hormones, corticosteroids, glucocorticoids, mineralocorticoids, and anabolic steroids. Particular sex hormone steroids that can be used include androgens, androstenediol, androstenedione, dehydroepiandrosterone, dihydrotestosterone, testosterone, estrogens, estradiol, estriol, estrone, progestogens, progesterone, or pharmaceutical equivalents thereof. Particular corticosteroids that can be used include Class A corticosteroids (including hydrocortisone acetate, methylprednisolone, prednisolone, cloprednol, fludrocortisone acetate, and pharmaceutical equivalents thereof), Class B corticosteroids (including amcinonide, desonide, fluocinolone acetonide, halcinonide, triamcinolone acetonide or diacetate, budesonide, or pharmaceutical equivalents thereof), Class C corticosteroids (including clocortolone pivalate, desoximetasone, betamethasone, dexamethasone, or pharmaceutical equivalents thereof), or Class D corticosteroids (including alclometasone dipropionate, betamethasone dipropionate, betamethasone valerate, clobetasol propionate, clobetasone butyrate, diflorasone diacetate, fluticasone propionate, mometasone furoate, or pharmaceutical equivalents thereof). The steroid can be isolated from biological material or synthesized.

Formulations of the present disclosure can then be prepared by adding a carrier to the steroid loaded mineral coated microparticles. In one embodiment, a carrier including an active agent can be added to mineral coated microparticles having the active agent adsorbed to the mineral coating to prepare a formulation including bound active agent (active agent adsorbed to the mineral coated microparticle) and unbound active agent. At least one of the active agents adsorbed to or incorporated within the mineral coated microparticle is a steroid. In another embodiment, a carrier not including an active agent can be added to mineral coated microparticles having the active agent adsorbed to the mineral to prepare a formulation including bound active agent.

In particularly suitable formulation embodiments, the formulations include both bound and unbound active agent. Without being bound by theory, it is believed that injection of a formulation including mineral coated microparticles with bound active agent, including a steroid, and unbound active agent allows the unbound active agent to provide an immediate effect whereas bound active agent is sequestered by its adsorption to the mineral coated microparticle and provides a sustained effect as the mineral coating degrades and releases the active agent.

In one embodiment, the carrier is a pharmaceutically acceptable carrier. Pharmaceutically acceptable carriers, and, optionally, other therapeutic and/or prophylactic ingredients can be “acceptable” in the sense of being compatible with the other ingredients of the formulation and not be harmful to the recipient thereof. Suitable pharmaceutically acceptable carrier solutions include water, saline, isotonic saline, phosphate buffered saline, Ringer's lactate, and the like. The compositions of the present disclosure can be administered to animals, preferably to mammals, and in particular to humans as therapeutics per se, as mixtures with one another or in the form of pharmaceutical preparations, and which as active constituent contains an effective dose of the active agent, in addition to customary pharmaceutically innocuous excipients and additives.

Formulations for parenteral administration (e.g. by injection, for example bolus injection or continuous infusion) can be presented in unit dose form in ampoules, pre-filled syringes, small volume infusion or in multi-dose containers with and without an added preservative. The formulations can take such forms as suspensions, solutions, or emulsions in oily or aqueous vehicles, and may contain formulation agents such as suspending, stabilizing and/or dispersing agents. Alternatively, the mineral coated microparticles with active agent may be in powder form, obtained for example, by lyophilization from solution, for constitution with a suitable vehicle, e.g. sterile, pyrogen-free water, before use.

In one aspect, the present disclosure is directed to a mineral coated microparticle comprising at least one active agent incorporated within a mineral coating and at least one active agent adsorbed to the mineral coating. At least one of the active agents is a steroid.

As disclosed herein, to incorporate the active agent(s) within the mineral coated microparticle, active agent(s) are included in the simulated body fluid during the mineral coating process. Particularly suitable active agents include those described herein.

As described herein, the active agent can be adsorbed to the mineral coating. The active agent can also be incorporated within the mineral of the mineral coated microparticle, as described herein. The active agent can further be adsorbed to the mineral coating and incorporated within the mineral of the mineral coated microparticle, as described herein. As also described herein, different active agents can be adsorbed to or incorporated within the mineral. At least one of the active agents is a steroid.

In another aspect, the present disclosure is directed to a method for immediate and sustained delivery of a steroid. The method includes providing a formulation to an individual in need thereof, the formulation including a mineral coated microparticle comprising core and a mineral coating on the core; and a steroid. In one embodiment, the steroid is adsorbed to the mineral coating. In another embodiment, the steroid is incorporated within the mineral coating. In another embodiment, multiple active agents are adsorbed onto or incorporated with the mineral coating. At least one or more of the active agents is a steroid. In one embodiment, multiple steroids are adsorbed and/or incorporated with the mineral coating.

The steroid can be any type of steroid including sex hormones, corticosteroids, glucocorticoids, mineralocorticoids, and anabolic steroids. Particular sex hormone steroids that can be used include androgens, androstenediol, androstenedione, dehydroepiandrosterone, dihydrotestosterone, testosterone, estrogens, estradiol, estriol, estrone, progestogens, progesterone, or pharmaceutical equivalents thereof. Particular corticosteroids that can be used include Class A corticosteroids (including hydrocortisone acetate, methylprednisolone, prednisolone, cloprednol, fludrocortisone acetate, and pharmaceutical equivalents thereof), Class B corticosteroids (including amcinonide, desonide, fluocinolone acetonide, halcinonide, triamcinolone acetonide or diacetate, budesonide, or pharmaceutical equivalents thereof), Class C corticosteroids (including clocortolone pivalate, desoximetasone, betamethasone, dexamethasone, or pharmaceutical equivalents thereof), or Class D corticosteroids (including alclometasone dipropionate, betamethasone dipropionate, betamethasone valerate, clobetasol propionate, clobetasone butyrate, diflorasone diacetate, fluticasone propionate, mometasone furoate, or pharmaceutical equivalents thereof). The steroid can be isolated from biological material or synthesized.

In one embodiment, the formulation includes a carrier for the mineral coated microparticles. In one embodiment, the carrier contains an active agent. In one embodiment, the steroid adsorbed to the mineral coating is the same as the active agent in the carrier. In another embodiment, the steroid adsorbed to the mineral coating is different from the active agent in the carrier. In another aspect, at least two different active agents are adsorbed to the mineral coating, one of which is a steroid. In one embodiment, all of the active agents adsorbed to and/or incorporated in the mineral coating are steroids.

Steroids can be sustainably delivered with formulations which include mineral coated microparticles and steroids as steroids are released in a continuous manner as the coating dissolves. Other active agents can also be sustainably delivered along with the steroid when adsorbed to or incorporated in the mineral coating. The mineral coated microparticles can be delivered in a carrier solution containing an active agent to improve sustained delivery of the steroid.

Suitable methods for administration of formulations of the present disclosure are by parenteral (e.g., intramuscular, subcutaneous, intraperitoneal, or local injection into a tissue) administration routes. Local injection of the formulation into a tissue can be used to locally delivery steroid to a site where it is needed while decreasing systemic exposure to the steroid which may have unwanted side effects. In one embodiment, the formulation is administered through local injection into the synovium. In another embodiment, the formulation in injected intra-articularly to deliver steroid to the synovial fluid and/or the synovial lining. In yet another embodiment the formulation is injected into an organ. In another embodiment, the formulation is injected into a site of injury. Oral administration can also be used as a route of administration for the formulation containing mineral coated microparticles and a steroid. Oral administration can be utilized for sustained delivery of steroids in tissue of the digestive track, including the esophagus, the stomach, the small and large intestines, and the colon. Oral administration of the formulation containing mineral coated microparticles and a steroid can also be used for systemic administration of steroids. Inhaled administration can also be for delivery of the formulation of mineral coated microparticles and steroids. Inhaled administration may be used to locally deliver steroid to the lung or systemically delivery steroids. Administration routes and the formulations administered ordinarily include effective amounts of product in combination with acceptable diluents, carriers and/or adjuvants. Standard diluents such as human serum albumin are contemplated for pharmaceutical compositions of the invention, as are standard carriers such as saline.

Sustained delivery of the active agent, including the steroid, can be determined to obtain active agent release values that mimic established therapeutic levels of the active agent. The mass of mineral coated microparticles (with the steroid included) required to deliver a desired concentration of the steroid over a period of time can be calculated beforehand. For example, a single bolus injection of the steroid that provides the desired therapeutic effect can be delivered in a sustained manner over the desired period of time by obtaining the steroid release values from the mineral coated microparticles. Then the mass of mineral coated microparticles needed to deliver the steroid to provide the therapeutic effect of a desired period of time can be calculated. The localized and sustained delivery platform offers the benefit of continuous therapeutic levels of the steroid at the injury site or the site of inflammation without the requirement for multiple injections.

Effective dosages are expected to vary substantially depending upon the steroid(s) and other active agents used and the specific disease, disorder, or condition treated. Because of the rapid and sustained delivery of the active agents contained in the formulations of the present disclosure, suitable dosages are expected to be less than effective dosages of active agents delivered via bolus injections. As described herein, mineral coated microparticles can be prepared to deliver an effective amount of the steroid over the course of several days. Thus, administration of formulations of the instant application provide a bolus administration of unbound active agent that has a rapid effect and the sustained release of the active agent(s), including at least one steroids, during degradation of the mineral coating of the mineral coated microparticle has a sustained release of the steroid to maintain the effect over the course of hours to days as desired.

Formulations of the present disclosure can be administered to subjects in need thereof. As used herein, “a subject” (also interchangeably referred to as “an individual” and “a patient”) refers to animals including humans and non-human animals. Accordingly, the compositions, devices and methods disclosed herein can be used for human and veterinarian applications, particularly human and veterinarian medical applications. Suitable subjects include warm-blooded mammalian hosts, including humans, companion animals (e.g., dogs, cats), cows, horses, mice, rats, rabbits, primates, and pigs, preferably a human patient.

As used herein, “a subject in need thereof” (also used interchangeably herein with “a patient in need thereof”) refers to a subject susceptible to or at risk of a specified disease, disorder, or condition. The methods disclosed herein can be used with a subset of subjects who are susceptible to or at elevated risk of inflammatory diseases and disorders. Because some of the method embodiments of the present disclosure are directed to specific subsets or subclasses of identified subjects (that is, the subset or subclass of subjects “in need” of assistance in addressing one or more specific conditions noted herein), not all subjects will fall within the subset or subclass of subjects as described herein for certain diseases, disorders or conditions.

In another aspect, the present disclosure is directed to a method for treating inflammation or inflammatory disease or condition in a subject in need thereof. The method includes administering a formulation to the subject, wherein the formulation includes a mineral coated microparticle and a steroid.

In some embodiments, the method is directed to locally treating osteoarthritis (OA). Local treatment of OA can involve administration of the formulation containing mineral coated microparticles and a steroid to the synovium, synovial fluid, synovial lining, or other tissue within a joint. Administration can be through local injection.

In some embodiments, the method is directed to locally treating inflammation following an injury. Local treatment of an injury can involve administration of the formulation containing mineral coated microparticles and a steroid directly to the site of injury. Alternatively, local treatment of an injury can involve administration of the formulation containing mineral coated microparticles and a steroid to a site adjacent and/or near the site of injury. Local Administration can be through local injection. Suitable injuries which can be treated include orthopedic injury, lower back/spine injuries, cardiac injury, neurological injury, and the like.

Other suitable inflammatory diseases include type 2 diabetes, autoimmune diseases, and neuropathic diseases (e.g., Alzheimer's disease) as well as local and acute inflammatory situations (e.g. cutaneous and ligament wound healing).

Corticosteroids are particularly useful steroids for treating inflammatory conditions Particularly useful corticosteroids include Class B corticosteroids, including Triamcinolone acetonide and pharmaceutically acceptable salts thereof. Class A (including prednisolone and pharmaceutical equivalents), Class C (including betamethasone and pharmaceutical equivalents), or Class D (fluticasone or fluticasone propionate and pharmaceutical equivalents) corticosteroids are also particularly useful.

In another aspect, the present disclosure is directed to a method for treating pain in a subject in need thereof. The method includes administering a formulation to the subject, wherein the formulation includes a mineral coated microparticle and a steroid.

In some embodiments, the method is directed to locally treating pain associated with osteoarthritis (OA). Local treatment of OA can involve administration of the formulation containing mineral coated microparticles and a steroid to the synovium, synovial fluid, synovial lining, or other tissue within a joint. Administration can be through local injection.

In some embodiments, the method is directed to locally treating pain associated with an injury. Local treatment of pain associated with an injury can involve administration of the formulation containing mineral coated microparticles and a steroid directly to the site of injury. Alternatively, local treatment can involve administration of the formulation containing mineral coated microparticles and a steroid to a site adjacent and/or near the site of injury. Local Administration can be through local injection. Suitable injuries which can be treated include orthopedic injury, lower back/spine injuries, cardiac injury, neurological injury, and the like.

In some embodiments, the method is directed to locally treating pain not associated with an injury. Local treatment of pain can involve administration of the formulation containing mineral coated microparticles and a steroid directly to the site of pain. Alternatively, local treatment can involve administration of the formulation containing mineral coated microparticles and a steroid to a site adjacent and/or near the site of pain. Local Administration can be through local injection. Suitable injuries which can be treated include orthopedic injury, lower back/spine injuries, cardiac injury, neurological injury, and the like.

The mineral coated microparticle formulation can be delivered to the patient periodically, including about once every 1, 2, 3, 4, 5, 6, 7, 8, 9, 10, 12, 14, 16, 18, 20, 30, 50, 70, 90, 120, 150, or 180 days.

### EXAMPLES

### Example 1

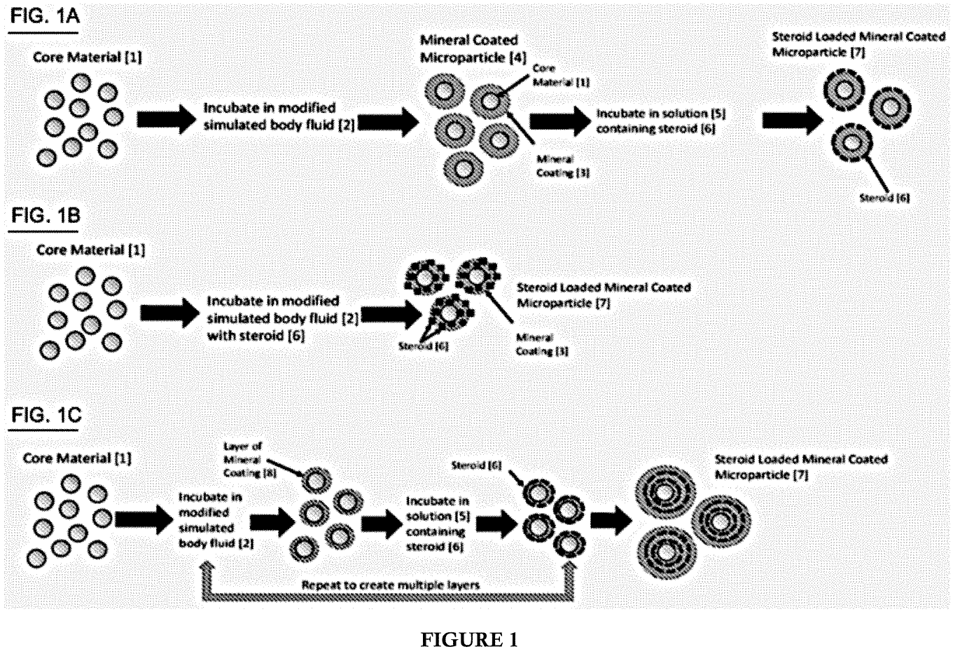

In this Example, mineral coated microparticles which adsorb steroid after coating formation is represented (FIG. 1A). A core material [1] is incubated in modified simulated body fluid [2] which contains the same concentration of ions as human blood plasma but with twice the concentration of calcium and phosphate. After incubating the core material in the modified simulated body fluid [2] for an extended period of time (usually between 3-14 days), a mineral coating [3] of sufficient thickness precipitates on the core [1] to form the mineral coated microparticle [4]. After coating, the mineral coated microparticles [4] are incubated in a solution [5] containing a steroid [6]. As a result, steroid [6] is adsorbed to the mineral coating [3] to form a steroid loaded mineral coated microparticle [7].

### Example 2

In this Example, mineral coated microparticles which incorporate steroid during coating formation is represented (FIG. 1B). A core material [1] is incubated in modified simulated body fluid [2] which contains the same concentration of ions as human blood plasma but with twice the concentration of calcium and phosphate and which also contains a steroid [6]. After incubating the core material in the modified simulated body fluid [2] and the steroid [6] for an extended period of time (usually between 3-14 days), a mineral coating [3] of sufficient thickness precipitates on the core [1] which incorporates steroid [6] throughout the mineral coating [3]. As a result, a steroid loaded mineral coated microparticle [7] is formed.

### Example 3

In this Example, mineral coated microparticles which incorporate steroid onto multiple layers of mineral coating is represented (FIG. 1C). A core material [1] is incubated in modified simulated body fluid [2] which contains the same concentration of ions as human blood plasma but with twice the concentration of calcium and phosphate. After incubating the core material in the modified simulated body fluid [2] extended period of time (usually between 1-3 days), a layer of mineral coating [8] of sufficient thickness precipitates on the core [1]. The resulting microparticle is incubated in a solution [5] containing a steroid [6]. After this the coating and steroid incubation steps are repeated to form multiple layers of mineral coating [8] with steroid [6] adsorbed. As a result, a steroid loaded mineral coated microparticle [7] is formed.

1-124. (canceled)

125. A mineral coated microparticle composition comprising a core enveloped in a mineral coating, and a steroid adsorbed onto or dispersed within the mineral coating, wherein the steroid is released upon dissolution of the mineral coating.

126. The mineral coated microparticle composition of claim 125, wherein the core comprises a polymer, ceramic, metal, glass or any combination thereof.

127. The mineral coated microparticle composition of claim 125, wherein the mineral coating comprises one or more layers of a calcium-phosphate, beta-tricalcium phosphate, and/or hydroxyapatite.

128. The mineral coated microparticle composition of claim 127, wherein the one or more layers of the calcium-phosphate mineral coating comprise a substitution ion.

129. The mineral coated microparticle composition of claim 128, wherein the substitution ion is chosen from carbonates and/or fluorides.

130. The mineral coated microparticle composition of claim 125, wherein the steroid is chosen from a sex hormone, a corticosteroid, a glucocorticoid, a mineralocorticoid, and an anabolic steroid.

131. The mineral coated microparticle composition of claim 125, wherein the mineral coating is nano-structured or porous.

132. A method for sustained delivery of a steroid to a subject in need thereof comprising: administering a mineral coated microparticle composition comprising a core enveloped in a mineral coating, and a steroid adsorbed onto or dispersed within the mineral coating, wherein the steroid is released upon dissolution of the mineral coating.

133. The method of claim 132, wherein the core comprises a polymer, ceramic, metal, glass or any combination thereof.

134. The method of claim 132, wherein the mineral coating comprises one or more layers of calcium-phosphate, beta-tricalcium phosphate, and/or hydroxyapatite.

135. The method of claim 132, wherein the one or more layers of the calcium-phosphate mineral coating comprise a substitution ion.

136. The method of claim 135, wherein the substitution ion is chosen from carbonates or fluorides.

137. The method of claim 132, wherein the mineral coated microparticle composition is administered by mouth, by subcutaneous injection, by inhalation, by topical application or by local injection to a site of inflammation.

138. The method of claim 132, wherein the steroid is released over a period of at least about 30 days.

139. The method of claim 132, wherein the sustained release of the steroid from the mineral coating improves the therapeutic efficacy of the steroid.

140. The method of claim 132, wherein the sustained release of the steroid from the microparticle increases the time between administrations of the mineral coated microparticle composition required to maintain therapeutic efficacy.

141. The method of claim 132, wherein the steroid comprises a Class A, Class B, Class C or Class D corticosteroid.

142. The method of claim 141, wherein
the Class A corticosteroid is chosen from hydrocortisone acetate, methylprednisolone, prednisolone, cloprednol, fludrocortisone acetate, or pharmaceutical equivalents thereof;
the Class B corticosteroid is chosen from amcinonide, desonide, fluocinolone acetonide, halcinonide, triamcinolone acetonide or diacetate, budesonide, or pharmaceutical equivalents thereof;
the Class C corticosteroid is chosen from clocortolone pivalate, desoximetasone, betamethasone, dexamethasone, or pharmaceutical equivalents thereof, and
the Class D corticosteroid is chosen from alclometasone dipropionate, betamethasone dipropionate, betamethasone valerate, clobetasol propionate, clobetasone butyrate, diflorasone diacetate, fluticasone propionate, mometasone furoate, or pharmaceutical equivalents thereof.

143. A method of reducing inflammation in a patient, comprising administering a mineral coated microparticle composition comprising a core enveloped in a mineral coating, and a steroid adsorbed onto or dispersed within the mineral coating, wherein the steroid is released upon dissolution of the mineral coating.

144. The method of claim 143, wherein the patient has arthritis and the mineral coated microparticle composition is administered by local injection into a joint.

145. (canceled)
